(19) United States
(12) Patent Application Publication
(10) Pub. No.: US 2012/0065625 A1
(43) Pub. Date: Mar. 15, 2012

(54) CATHETER/PUMP CONNECTOR WITH GUIDE SURFACE AND SYSTEM/METHOD FOR USING SAME

(76) Inventor: Brian D. Nelson, Birchwood, MN (US)

(21) Appl. No.: 13/227,765

(22) Filed: Sep. 8, 2011

Related U.S. Application Data
(60) Provisional application No. 61/382,354, filed on Sep. 13, 2010.

(51) Int. Cl. A61M 25/16

(52) U.S. Cl. 604/533

(57) ABSTRACT

Connectors and methods for connecting medical tubing (e.g., catheters) to an implantable infusion pump are described herein. In some embodiments, the connectors may include a housing having an internal guide surface that assists in guiding the connector onto a stem port received by the connector when the connector is misaligned with the stem port.

### RELATED APPLICATION(S)

This application claims the benefit of U.S. Prov. Pat. Appl. No. 61/382,354, filed Sep. 13, 2010, the content of which is incorporated herein by reference in its entirety.

### TECHNICAL FIELD

Embodiments of the invention described herein include implantable medical devices and systems, and more particularly, connectors, systems, and methods for coupling medical tubing (e.g., a catheter) to an implantable device such as a drug infusion pump.

### BACKGROUND

Treatment of diseases and ailments of the body often benefit from short- or long-term infusion of drugs and/or other fluids. While such medications may be administered extracorporeally, e.g., via transcutaneous injection, many patients benefit from the consistent and repeatable dosage provided by an implanted drug infusion pump. Such pumps may be used in a variety of applications such as control of pain and/or spasticity. They are well-suited to deliver infusate fluids to a targeted delivery site such as an epidural or intrathecal space of the spinal canal, or a particular location within the brain.

Drug infusion pumps are typically implanted subcutaneously, e.g., in the chest or abdominal cavity. The pump may incorporate a chamber to hold the infusate fluid. A needle-penetrable septum may also be provided and is preferably located generally directly beneath the skin. The septum allows drugs or other fluids to be introduced into the infusate chamber by transcutaneous injection. The pump may also include a relatively rigid fluid discharge outlet or stem through which the drug is directed during delivery. The outlet is typically connected to flexible medical tubing, e.g., a catheter, via a pump connector.

A secure and leak-free connection of the catheter to the pump outlet is beneficial to ensure correct dosage delivery to the targeted delivery site. If the connection should somehow fail, the intended infusate dosage may not reach the delivery site and, moreover, some (or all) of the infusate could undesirably be dispensed in the vicinity of the pump outlet.

As may be appreciated by those skilled in the art, the pump, catheter, and connector components may be relatively small. While beneficial to implantation, the small size of these parts may present challenges if the parts are not adequately aligned prior to connection. For example, if the connector/catheter is skewed or otherwise “off-axis” prior to attachment with the pump outlet, the relatively rigid tip of the outlet may inadvertently cut or core an inner surface (e.g., the relatively soft catheter body or connector seal) of the connector/catheter.

### SUMMARY

Embodiments of the present invention may be directed to connectors, systems, and methods for connecting medical tubing (e.g., catheters) to a fluid delivery device such as an implantable medical device. In one embodiment, a medical system is provided including: a fluid delivery device comprising a protruding stem; and a catheter assembly. The catheter assembly includes: a catheter body defining a lumen; a seal attached at or near a proximal end of the catheter body, the seal defining a seal opening in fluid communication with the lumen; a connector surrounding the seal, the connector configured to secure an end of the catheter assembly to the stem; and a guide ring surrounded by the connector and located between the seal and an end face opening of the connector. The guide ring includes a tapered inner surface configured to contact the stem and align a centerline of the seal opening with a centerline of the stem when the stem is received into the end face opening of the connector.

In another embodiment, a medical system is provided that includes: an implantable medical device comprising a protruding stem; and a catheter assembly. The catheter assembly includes: a catheter body comprising a first end defining a ring-shaped seal, the seal defining a seal opening configured to receive therein the stem; a tubular connector configured to surround at least the seal, the connector comprising a first end defining an end face opening, the seal being spaced-apart from the end face opening of the connector; and an internal guide surface positioned within the connector between the end face opening and the seal. The guide surface includes a material resistant to deformation or damage upon contact with the stem of the medical device. The guide surface is configured to align a centerline of the stem, upon insertion of the stem into the end face opening of the connector, with a centerline of the seal opening.

In yet another embodiment, a method for connecting a catheter to a medical device is provided, wherein the method includes receiving a stem of the medical device into an opening of a connector attached to a first end of the catheter. The connector includes a housing having a first end defining the opening, the housing surrounding a ring-shaped seal spaced-apart from the first end, the seal attached to the catheter and defining a seal opening configured to receive therein the stem of the medical device. The connector also includes a tapered guide surface positioned within the housing between the first end and the seal. The method further includes introducing the stem of the medical device into the opening of the housing such that a longitudinal axis of the stem is misaligned from a longitudinal axis of the connector; contacting the stem of the medical device with the guide surface; and sliding the stem along the guide surface until the longitudinal axis of the connector is aligned with the longitudinal axis of the stem.

The above summary is not intended to describe each embodiment or every implementation of the present invention. Rather, a more complete understanding of the invention will become apparent and appreciated by reference to the following Detailed Description of Illustrative Embodiments in view of the accompanying figures of the drawing.

### BRIEF DESCRIPTION OF THE VIEWS OF THE DRAWING

The present invention will be further described with reference to the figures of the drawing, wherein:

FIGS. 1A and 1B illustrate implanted medical infusion pump systems incorporating a catheter/pump connector in accordance with embodiments of the present invention; wherein FIG. 1A illustrates a brain infusion system; and FIG. 1B illustrates a spinal infusion system;

FIG. 2 is an enlarged view of the infusion pump and catheter/pump connector of FIGS. 1A and 1B;

FIG. 3 is an enlarged view of the catheter/pump connector of FIG. 2 detached from the pump;

FIG. 4 is an exploded view of the connector of FIG. 3;

FIG. 5 is an enlarged view of an exemplary retaining member and housing of the connector of FIGS. 2 and 3 as assembled;

FIG. 6 is a diagrammatic end view of the retaining member of FIG. 5, wherein the retaining member is shown in a first or unactuated position A, and a second or actuated position B;

FIG. 7 is a section view of the connector of FIG. 3;

FIG. 8 is a section view of the connector of FIG. 3 as attached to a stem of the infusion pump;

FIG. 9 is a perspective view of a guide member in accordance with one embodiment of the invention; and

FIG. 10 is an enlarged section view of the guide member of FIG. 9.

Unless stated otherwise herein, the figures of the drawing are rendered primarily for clarity and thus may not be drawn to scale.

### DETAILED DESCRIPTION OF ILLUSTRATIVE EMBODIMENTS

In the following detailed description of illustrative embodiments, reference is made to the accompanying figures of the drawing that form a part hereof, and in which are shown, by way of illustration, specific embodiments of catheter/pump connectors, systems, and methods described herein. It is to be understood that other embodiments may be utilized and structural changes may be made without departing from the scope of the present invention.

In general, apparatus, systems, and methods described herein may be used to connect medical tubing, e.g., catheters, to a fluid source device such as a medical device. Preferably, the connectors, systems, and methods described herein provide substantially leak-free and secure coupling of the catheter to the device, yet may readily permit disconnection and removal by a physician when desired.

It is noted that the terms “comprises” and variations thereof do not have a limiting meaning where these teens appear in the accompanying description and claims. Moreover, “a,” “an,” “the,” “at least one,” and “one or more” are used interchangeably herein.

Relative terms such as left, right, forward, rearward, top, bottom, side, upper, lower, horizontal, vertical, and the like may be used herein and, if so, are from the perspective observed in the particular figure. These terms are used herein only to simplify the description, however, and not to limit the scope of the invention in any way.

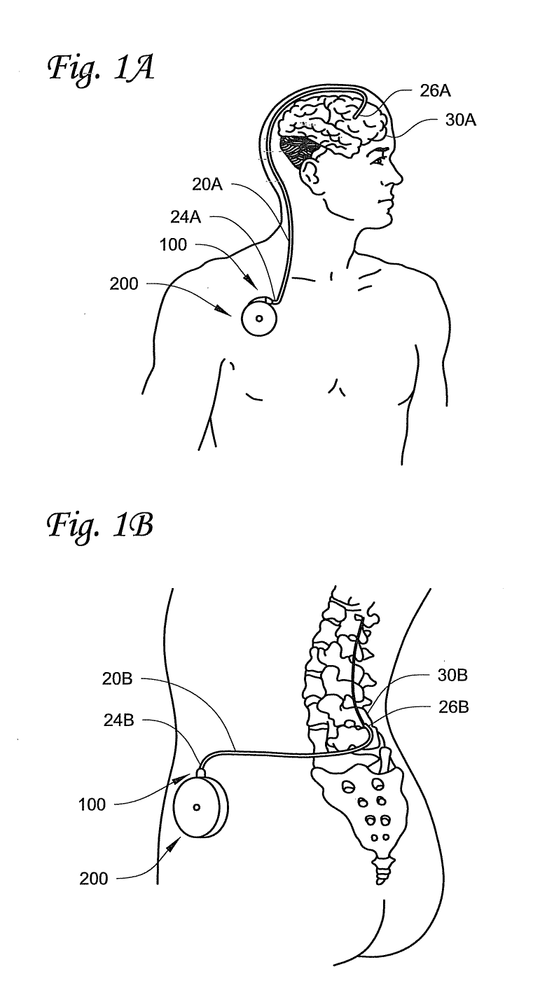

FIGS. 1A and 1B illustrate exemplary implantable medical systems that may utilize a connector and method in accordance with embodiments of the present invention. FIG. 1A illustrates a fluid delivery or other medical device, e.g., an implantable drug infusion pump 200 (e.g., an ISOMED or SYNCHROMED infusion pump sold by Medtronic, Inc. of Fridley, Minn., USA), configured to deliver an infusate drug to a specific location within the brain 30A. A catheter, e.g., catheter 20A, may include a proximal end 24A coupled to the pump 200, and a distal end 26A positioned near the targeted delivery site in the brain 30A. A connector 100 as described herein may be used to couple the catheter 20A to the pump 200. While a pump 200 is provided in the embodiment shown, the pump could be replaced with a variety of other implantable or external medical devices.

FIG. 1B illustrates another exemplary infusion system wherein a distal end 26B of a catheter, e.g., catheter 20B, is positioned within a spinal column 30B. The proximal end 24B is, once again, coupled to the pump 200 with a connector as described herein, e.g., the tubular connector 100.

While the exact size and construction of each catheter 20A and 20B (generically referred to herein as “catheter 20”) may vary, each catheter may, in one embodiment, be formed from extruded silicone tubing. In the illustrated applications, the catheter 20 may have an undeflected outer diameter of, e.g., about 1 to about 3 millimeters (mm). Other exemplary medical tubing materials may include polyurethane and blends and co-extrusions such as silicone/polyurethane.

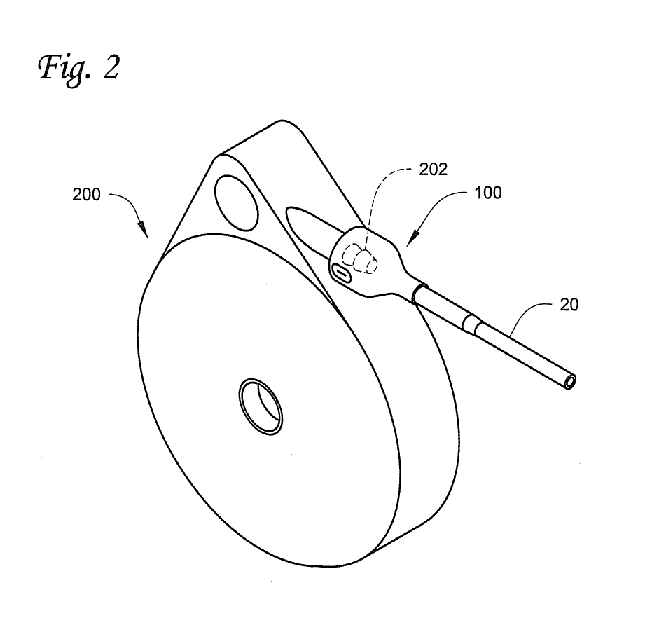

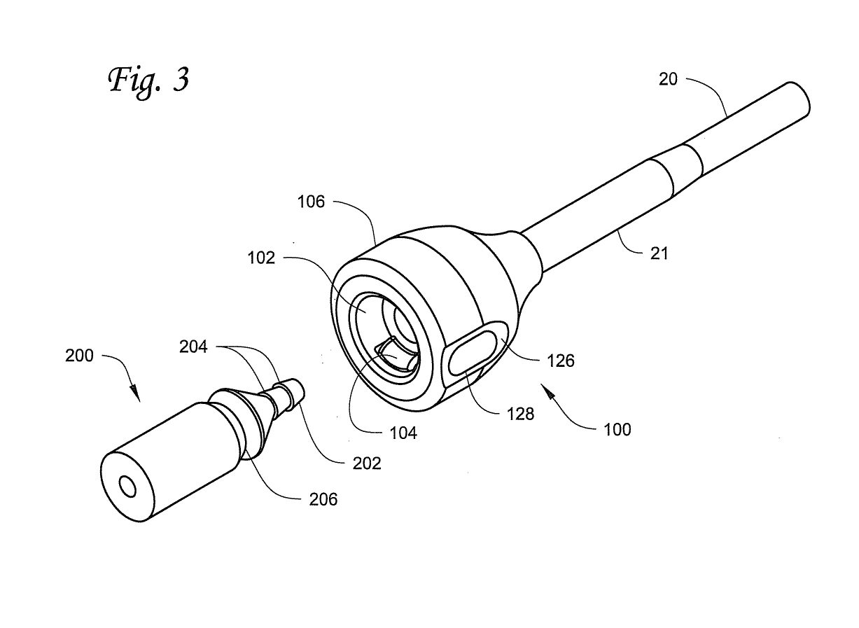

FIG. 2 illustrates an enlarged perspective view of the pump 200 and connector 100 of FIGS. 1A and 1B. Although not fully illustrated in FIG. 2, the pump 200 may include a discharge port or stem 202 (see also FIG. 3) to which the catheter 20 attaches and through which infusate from the pump is delivered. The stem 202 may define a protruding male stem forming a tapered cylinder with raised barbs 204 thereon (see FIG. 3), yielding a “Christmas tree” shape to better engage an inner surface of the catheter and/or seal as further described below.

As FIG. 3 illustrates, the catheter 20 (e.g., the proximal the end that attaches to the pump 200) may form the catheter body of a catheter assembly that may also include: a tubular seal 21 attached thereto (e.g., via mechanical capture (e.g., by providing protruding ribs on the catheter body exterior that engage corresponding recesses formed on an inner surface of the seal), adhesive, press fit, or the like), and the connector 100. In some embodiments, the seal 21 could be over-molded with the catheter (e.g., silicone over silicone catheter) or otherwise formed as an integral portion thereof. FIG. 3 illustrates other components that are described in more detail below.

Suitable materials for the seal 21 may include molded silicone or polyurethane. Preferably, the seal is elastic and relatively flexible (as compared to the housing 102 described further below) and attached at or near a proximal end of the catheter. However, use of other materials for the seal, including those that are more rigid, is possible.

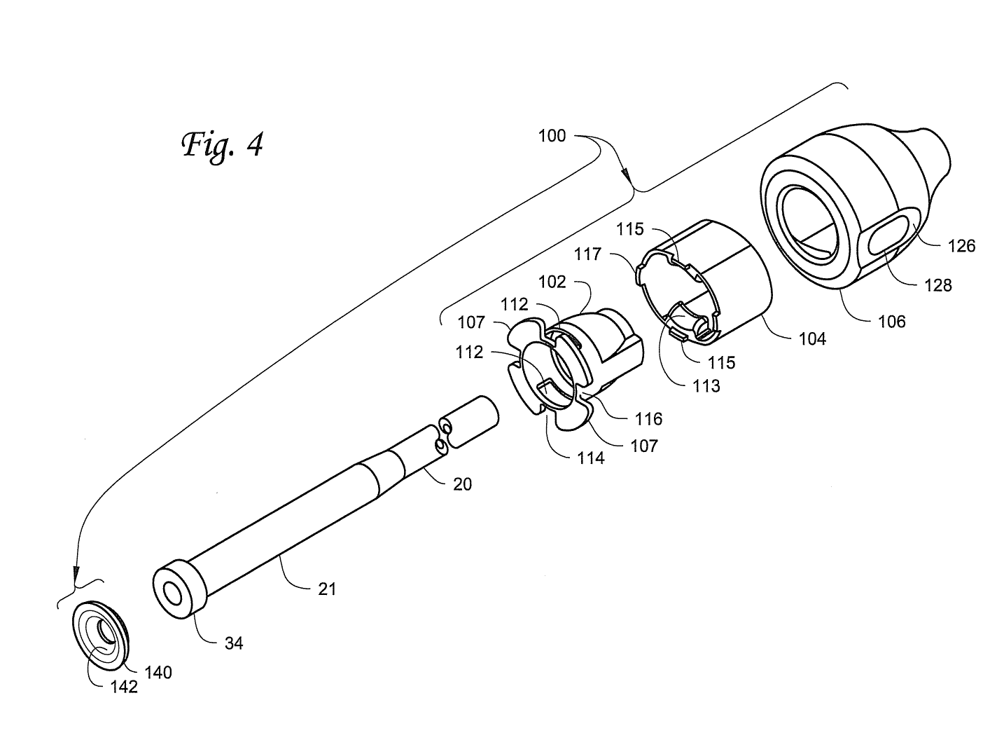

FIG. 4 is an exploded view of the exemplary catheter connector 100.

As clearly illustrated in this view, the connector 100 may include a retaining member 104 and a housing 102 that surrounds at least the seal 21. When the stem 202 is adequately coupled to the connector 100 as further described below, one or more locking members or fingers 113 of the retaining member 104 may engage a receiving portion of the stem (e.g., a groove 206 foamed in the stem 202 as shown in FIG. 3), thereby securely coupling the catheter assembly to the stem 202 of the pump 200.

An optional cover member, e.g., boot 106, may slide axially to cover the housing 102 and retaining member 104 as indicated in FIG. 3. The boot 106 may be configured to reduce stress on surrounding tissue by providing a relatively smooth transition surface around the connector 100. The boot 106 may also reduce or prevent excessive tissue growth within the internal parts of the connector 100. The boot 106 may, in one embodiment, be made of silicone rubber, e.g., white ETR silicone, and may optionally be loaded (e.g., 12.5% Barium Sulfate loaded or 2% Titanium Dioxide loaded) to give the connector a degree of radiopaqueness. While other materials are certainly possible, the use of silicone rubber provides the connector with a soft, impact-absorbent exterior.

While the connector 100 is illustrated herein as operable to couple the stem 202 of the implantable medical device (e.g., pump 200) to an end of the catheter assembly (e.g., to a female end of the catheter 20), such a configuration is not limiting. For example, the connector 100 could also be utilized with reversed configurations, e.g., an implantable drug infusion pump having a female-configured end (e.g., similar to the illustrated end of the catheter 20), while the catheter could include a male-configured stem (e.g., similar to the illustrated stem 202).

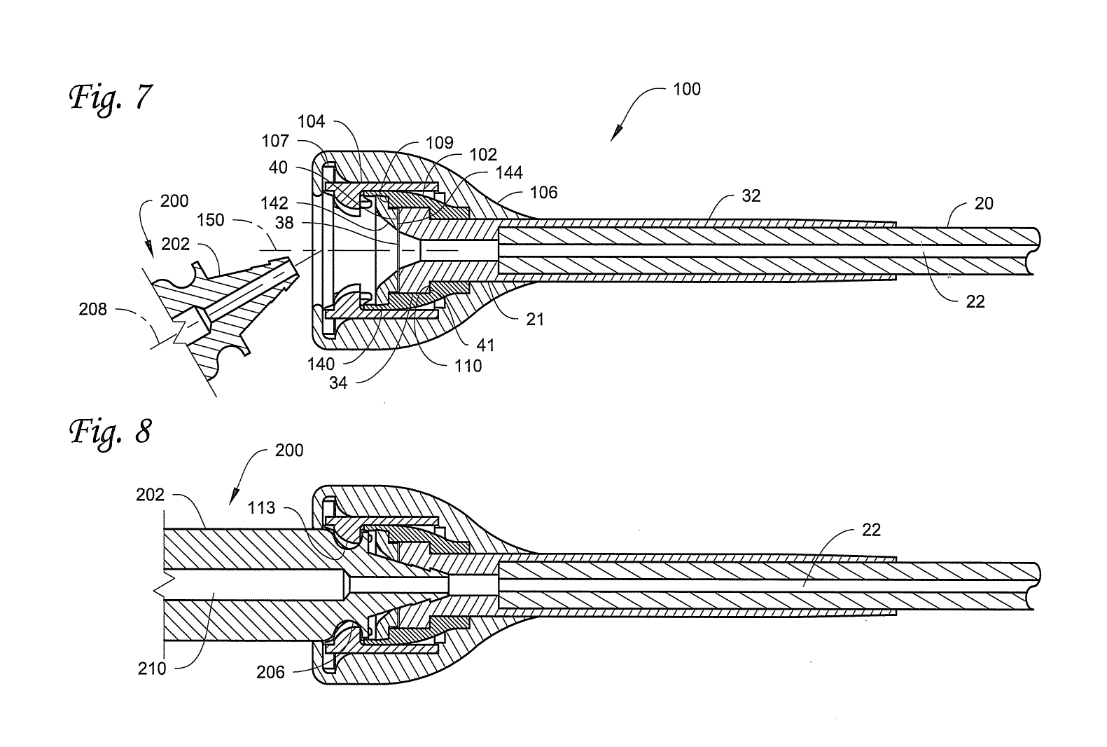

The catheter connectors described herein are similar in many respects to the catheter connectors described in U.S. Pat. No. 7,387,624 (Nelson), which is incorporated herein by reference in its entirety. The catheter connectors described herein do, however, include a housing 102 that includes an internal guide surface 142, e.g., a guide ring 140, as seen in the exploded perspective view of FIG. 4. The internal guide ring 140 depicted in FIG. 4 may be surrounded by the connector. That is, the guide ring 140 may be positioned within a stepped lumen of the connector housing 102 between an end face opening (see FIG. 3) of the connector (end face opening formed through both the retaining member and the housing) and the seal 21, the latter which may be spaced-apart from the end face opening of the connector. In the illustrated embodiment, the guide ring/guide surface is positioned adjacent to or proximate the seal (see, e.g., FIG. 7).

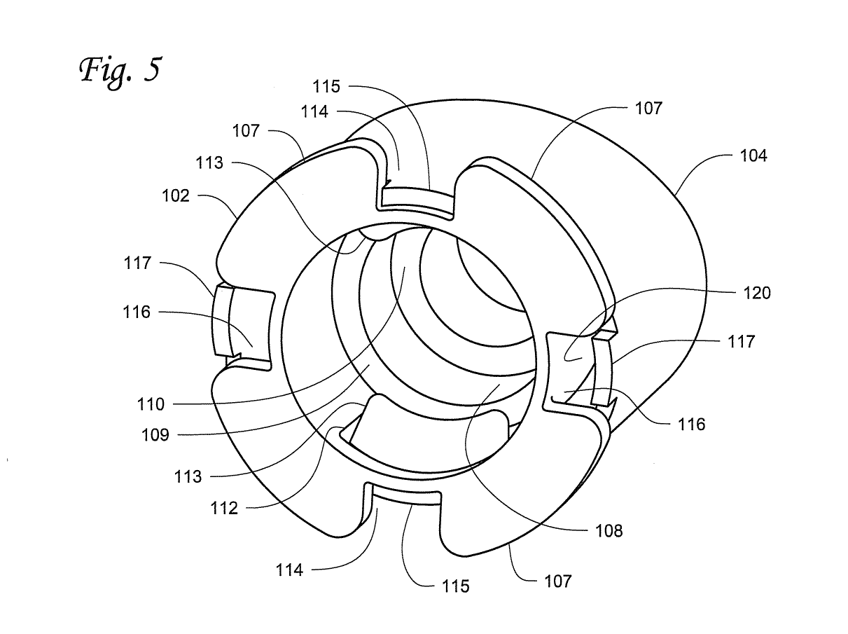

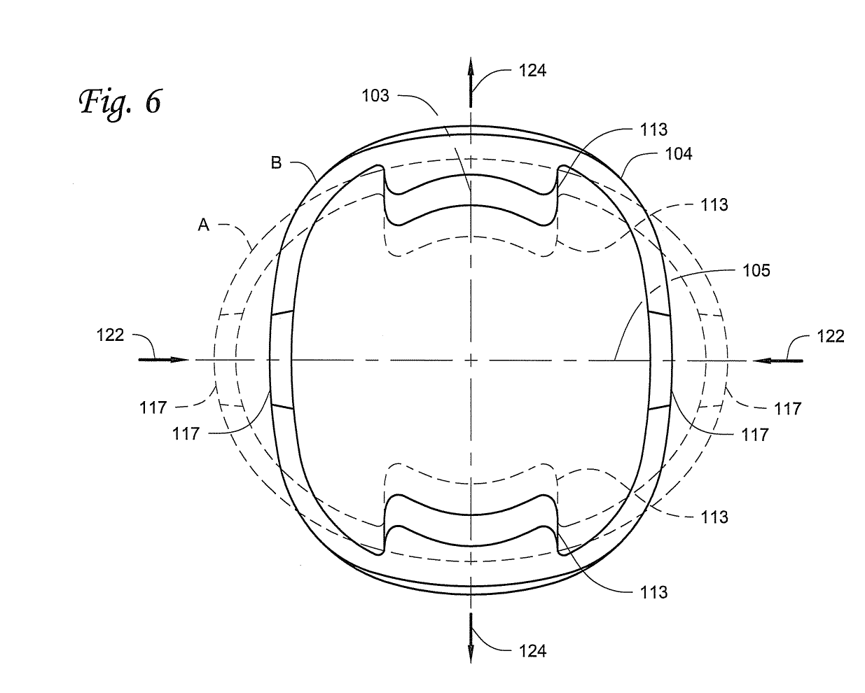

FIGS. 5 and 6 illustrate the general operation of the connector. FIG. 5 is an enlarged perspective view of an assembled portion or subassembly of the connector 100 of FIG. 4. This subassembly, e.g., connector body, may include, in one embodiment, the female retaining member 104 and the housing 102 (e.g., the guide ring 140 and catheter 20 are not shown in this view).

The housing 102 may include a tubular wall of a generally circular cross-section, and may include a housing flange 107 at or near the open end (“cross-section,” as used herein, indicates a section view taken along an identified line of sight or, where no line of sight is identified, along a conventional, e.g., longitudinal, axis of the referenced component). In the illustrated embodiment, a stepped lumen 108 extends through the housing, wherein the steps define one or more surfaces, e.g., abutting surfaces. For example, the steps may define a first abutting surface 109 and a second abutting surface 110. The abutting surfaces 109, 110 may contact corresponding surfaces of the guide ring 140 and seal 21, respectively, as described in more detail below.

The housing 102 may further include one or more slots. In the illustrated embodiments, these slots are formed in the housing flange 107 as shown in FIG. 5. For instance, the housing 102 may include first or vertical slots 114 positioned along a minor axis 103 (see FIG. 6) of the retaining member 104, and second or horizontal slots 116 positioned along the retaining member's major axis 105. The slots 114 and 116 may receive tabs, e.g., vertical tabs 115 and horizontal tabs 117, respectively, of the retaining member 104. The tubular housing 102 may further include one or more windows 112 (only one visible in FIG. 5) through which the one or more locking fingers 113 associated with the retaining member may selectively protrude as further described below. In the illustrated embodiment, there are two windows on opposing sides of the housing 102, and two locking fingers 113 on associated opposing sides of the retaining member 104.

In the illustrated embodiments, the housing 102 is preferably made of a relatively rigid, biocompatible material such as titanium, stainless steel, or rigid plastic.

FIG. 6 illustrates an end view of the retaining member 104, which, as shown in FIG. 5, surrounds the housing 102 during use. In the illustrated embodiment, the retaining member is deflectable and is shaped such that, when undeflected and viewed along its longitudinal axis (or in cross-section) as shown in FIG. 6, it defines the minor axis 103 and the major axis 105. The major axis 105 may span across the widest cross-sectional opening of the retaining member 104, while the minor axis 103, which is preferably orthogonal to the major axis 105, may span across a narrower portion, e.g., the smallest cross-sectional opening. While a variety of shapes may yield this configuration, the retaining member 104 is, in one embodiment, configured as a tubular sleeve with a generally elliptical cross-section (e.g., when viewed along its longitudinal axis) as shown in FIGS. 4-6. As a result, the greatest dimension or diameter of the ellipse (e.g., a line extending through the foci) defines the major axis 105.

As described above, the retaining member 104 may further include one or more locking fingers 113 protruding or extending inwardly from the tubular sleeve. The locking fingers 113 are preferably positioned along the minor axis 103 such that they pass through the corresponding windows 112 of the housing 102 (see FIG. 5) and selectively engage a receiving portion of the stem 202 (e.g., the groove 206 formed in the stem; see, e.g., FIG. 3) of the pump 200 as further described below.

The retaining member 104 may further include the vertical and horizontal tabs 115 and 117 that correspond in location to the vertical and horizontal slots 114 and 116 of the housing, respectively (see FIG. 5). That is, the vertical tabs 115 may be positioned along the minor axis 103, while the horizontal tabs 117 may be positioned along the major axis 105 (for clarity, the vertical tabs 115 are not illustrated in FIG. 6).

The increased width of the retaining member 104 along its major axis 105 results in a clearance between the retaining member 104 and the generally cylindrically shaped (e.g., circular cross-section) housing 102 along the major axis. For example, when the retaining member is in a first or unactuated (e.g., relaxed) position A as shown in FIG. 5, little or no clearance exists between the minor axis 103 of the retaining member 104 and the housing, i.e., in the vicinity of the vertical tabs 115. However, when in this unactuated position, a gap 120 preferably exists along the major axis 105 between the retaining member 104 and the housing, i.e., in the vicinity of the horizontal tabs 117. The gap 120 is beneficial as it provides space for compression of the retaining member 104. That is, the gap 120 allows the retaining member to elastically deflect from the first unactuated position A to a second actuated position B (see FIG. 6) without interference from the housing 102. Such actuation permits disengagement of the connector 100 (and thus the catheter) from the pump 200.

To actuate the retaining member 104, a horizontal compressive force 122 may be applied near the horizontal tabs 117, e.g., along the major axis 105 of the retaining member 104. The horizontal compressive force 122 may be applied by “squeezing” the retaining member, for example, between a physician's thumb and forefinger. As the retaining member 104 is compressed, the locking fingers 113 move outwardly (relative to the first position A) along the minor axis 103 (e.g., move in the direction indicated by arrows 124 in FIG. 6). As a result, the locking fingers 113 move from the first unactuated position A outwardly through the windows 112 (see FIG. 5).

The fingers 113 need only withdraw sufficiently to disengage from the stem. That is, when the retaining member is in the first unactuated position A, the locking fingers protrude inwardly through the window a first distance, while, when in the second actuated position B, the locking fingers may either: withdraw from the window; or protrude inwardly through the window a second distance that is less than the first distance.

In the illustrated embodiment, the gap 120 (see FIG. 5) permits elastic deflection of the retaining member 104 horizontally inwardly about 1 mm on each side. This movement results in vertical outward displacement of each locking finger 113 (away from the housing) of about 0.5 to about 0.7 mm, e.g., about 0.64 mm. The relatively rigid housing 102 may act as a stop, preventing the physician from over-squeezing and permanently deforming or breaking the retaining member 104.

The retaining member 104 is preferably made from a relatively flexible material to permit snap-fit engagement between the locking fingers 113 and the windows 112 of the housing. Exemplary materials may include injection-molded nylon (e.g., Grilamid TR55 Nylon 12 produced by EMS-Grivory), polysulfone (e.g., Udel Polysulfone P-1700 produced by Solvay Advanced Polymers, L.L.C.), or other engineering plastics.

The interrelation of the vertical and horizontal tabs 115, 117 of the retaining member 104 with the vertical and horizontal slots 114, 116 of the housing 102 assist in properly aligning the vertical and horizontal axes of the two components, relative to one another, during actuation. For example, the engagement of the vertical tabs 115 with the vertical slots 114 assists in maintaining the locking fingers 113 in alignment with the windows 112. Similarly, the engagement of the horizontal tabs 117 with the horizontal slots 116 assists in maintaining substantially equivalent displacement of each locking finger 113. Without the tabs 117 and slots 116, one locking finger 113 could remain stationary while the remaining finger could move twice its intended displacement. While the horizontal and vertical tabs provide these desirable alignment benefits, other embodiments may eliminate some or all of these tabs without departing from the scope of the invention. Still further, while the housing 102 is beneficial for reasons described herein, other embodiments may eliminate the housing altogether without departing from the scope of the invention.

FIG. 7 illustrates a cross-sectional view of the exemplary connector 100 as it is used to couple the catheter 20 to the stem 202 of the infusion pump 200. In this view, the stem is 202 is depicted as if the connector is initially introduced obliquely to the stem, e.g., a centerline or longitudinal axis 208 of the stem is not first aligned with a centerline or longitudinal axis 150 of the seal/connector. As clearly illustrated in this view, one or both of the housing and retaining member define generally female (e.g., tubular or sleeve-shaped) members having a first end defining a first opening.

As described above, the catheter 20 may include the seal 21, wherein the seal may include both an elongate body portion 32, and an elastic, ring-shaped flange portion 34 defined by a generally cylindrical but stepped surface. In the illustrated embodiment, the catheter 20 may extend proximally into the body portion 32 as shown. In other embodiments, the catheter may extend farther, e.g., into the flange portion 34 of the seal 21. A second or seal opening or lumen, which may be tapered, may extend from a face 38 of the seal 21 to a lumen 22 of the catheter 20 such that the seal opening is in fluid communication with the lumen 22. The seal lumen may be configured to receive and seal against the male stem 202 (see, e.g., FIG. 8). As a result, when the seal 21 is coupled to the stem 202, the lumen 22 of the catheter 20 may be in fluid communication with a lumen 210 (see also FIG. 8) of the pump, e.g., in communication with an infusate chamber of the pump 200.

The embodiment of the internal guide ring 140 depicted in FIG. 7 fits within the first opening or lumen defined by the housing 102. The internal guide ring 140 may be positioned within the housing 102 (e.g., along an inner surface of the housing) such that the guide ring 140 nests within the lumen and is seated against the first abutting surface 109. The guide ring 140 preferably includes a guide surface 142 operatively facing the interior of the lumen of the housing 102. The guide surface 142 may define a tapered inner surface defining a centerline that is coaxial with the centerline 150 of the seal opening. The tapered inner surface may further taper from a large diameter or opening to a small diameter or opening, wherein the small diameter is located proximate the seal. The guide surface 142 preferably acts to contact and align the stem 202 (or other port member received into the connector housing 102) with the center of the lumen defined by seal 21/housing 102. Stated alternatively, the guide surface 142 may align the longitudinal axis 150 of the seal opening with the longitudinal axis 208 of the stem 202 when the latter is received within the end face opening of the connector.

Although the guide surface 142 of the depicted internal guide ring 140 is generally frusto-conical as seen in the cross-sectional views of FIGS. 7 and 8, the guide surfaces in other embodiments may take other forms, e.g., ramp, arcuate. Still other guide surfaces may include combinations of both arcuate portions and flattened portions. For example, as shown in FIGS. 7, 9, and 10, the guide surface may have a curved inlet in addition to the frusto-conical surface.

The internal guide ring 140, like the housing 102, may preferably be made of a relatively rigid, biocompatible material such as titanium, stainless steel, or rigid plastic. In the illustrated embodiment, the guide ring 140 is made of a material that is resistant to deformation or damage (e.g., puncture, scraping) upon forced contact with the stem 202. Thus, it is preferred that the guide ring 140 be constructed of a material or materials that are different, e.g., harder or more rigid, than the materials used for catheter and seal 21. The materials used for the housing 102 and the guide ring 140 may be the same or different.

The housing 102 and the guide ring 140 may be produced by most any acceptable manufacturing process, e.g., machining, injection molding, etc. Although depicted as two separate components, in some embodiments, the housing and the guide ring may be constructed as a one-piece, completely integral component, in which case the housing may be described as having an integral guide surface positioned within the lumen of the housing.

The flange portion 34 of the seal 21 may form one or more surfaces, e.g., first surface 40 and second or compression surface 41. The first surface 40 and the second surface 41 may be, e.g., flat surfaces oriented normal to the longitudinal axis 150 extending through the connector. That is, one or both of the surfaces 40 and 41 may lie within a plane perpendicular to the centerline 150 of the seal 21. In the depicted embodiment, the first surface 40 is found at the proximal end (e.g., face 38) of the flange portion 34 of the seal 21, while the second surface 41 is found at a distal or opposite end of the flange portion 34.

When the guide ring is installed as shown in FIG. 7, the first surface 40 may be located adjacent to a distal facing surface 144 of the guide ring 140, while the second or compression surface 41 typically contacts the abutting surface 110 of the connector housing 102. Contact between the facing surfaces of the seal 21 and the guide ring 140 is not necessary as the actual fluidic seal occurs within the lumen of the seal itself (see FIG. 8). However, in one embodiment, the flange portion 34 of the seal could be compressed within the housing 102 between the distal facing surface 144 of the guide ring 140 and the proximal facing abutting surface 110 of the housing 102 such that no gap exists between the seal and the guide ring. Such compression of the seal could remove potential play between the various components.

Moreover, while illustrated as incorporating the seal 21 (e.g., flange portion 34), other catheter embodiments lacking this feature are also contemplated. For example, a catheter having a tapered end could be substituted without departing from the scope of the invention, provided that the catheter and the housing were arranged to provide an adequate, fluid-tight seal.

During use, the retaining member 104 and housing 102 may be preassembled (to the arrangement seen in FIG. 5) and slid over a distal end of the catheter 20 and seal 21 until the abutting surface 110 is near or in contact with the compression surfaces 41 of the seal. The optional boot 106 may then be slid over the distal end of the catheter 20 and positioned a short distance from the housing 102 and the retaining member 104.

The guide ring 140 may be inserted, e.g., press-fit, into and seated within the housing 102 after the seal 21 is positioned. In other embodiments, the guide ring may be attached via other methods, e.g., adhesive or laser-welding. Insertion of the guide ring 140 into the housing 102 may retain, and optionally partially compress, the flange portion 34 of the seal 21 between the surface 144 of the guide ring 140 and the surface 110 of the housing 102.

With the housing 102 and related components assembled as seen in

FIG. 7, the protruding stem 202 may then be received into the connector, e.g., into the end face opening and the lumen of the flange portion 34 of the seal 21, such that at least a portion of the stem is surrounded by the connector housing 102 as shown in FIG. 8. As indicated in FIGS. 7 and 8, the guide surface 142 of the guide ring 140 may guide or redirect the stem 202 (if it is inserted into the connector obliquely or otherwise along an off-axis or misaligned path) towards a center of the housing e.g., towards the lumen of the seal. This aligning function may occur when a tip of the skewed stem contacts the guide surface 142 and slides along the guide surface until the longitudinal axis 150 of the connector is aligned with the longitudinal axis 208 of the stem. To provide this functionality, the guide surface 142 is preferably sized and located within the housing to minimize or even prevent contact of the stem directly with the seal.

The stem 202 may be inserted until the tip of the stem enters the lumen of the seal. In some embodiments, the catheter 20 could extend into the seal itself such that the stem may ultimately be positioned within the lumen 22 of the catheter. When the stem 202 is fully received in the housing 102, the locking fingers 113 may engage the receiving portion 206 of the stem as described above.

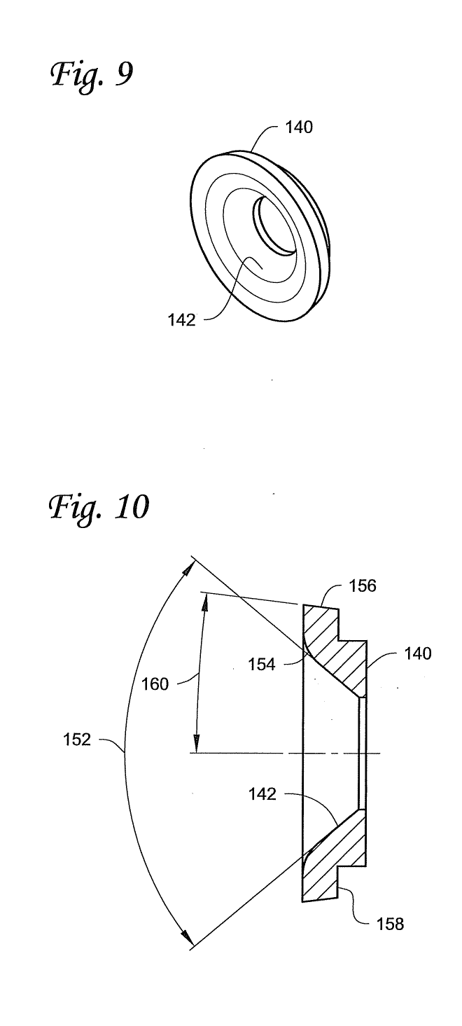

FIGS. 9 and 10 illustrate an exemplary embodiment of the guide ring 140. As shown in these figures, the guide ring may include the frusto-conical surface 142, which may, in one embodiment, define a cone angle 152 of about 70 degrees to about 90 degrees, e.g., about 80 degrees. Moreover, the opening to the frusto-conical surface 142 may be rounded or chamfered, e.g., it may have a rounded edge 154 as shown in FIG. 10.

The guide ring 140 may also include an outer surface 156 configured to seat with interference (e.g., a press fit) against an inner surface of the housing 102. To assist with press-fitting the guide ring into the housing, the surface 156 may be tapered as compared to the inner surface of the housing. In one embodiment, the taper angle 160 is about 5 degrees to about 10 degrees, e.g., about 7 degrees, although other taper angles are certainly possible. The guide 140 ring may further include a surface 158 which contacts the first abutting surface 109 (see FIG. 7) when the guide ring is fully inserted therein.

As already discussed herein, upon receipt of the stem 202 (see FIG. 8), the locking fingers 113 of the retaining member 104, which extend through the windows 112 of the housing 102, may deflect towards their second positions as they pass over the lip that defines the groove 206 as indicated in FIG. 8. Once the locking fingers reach the groove 206 of the stem 202, they may return to their first (undeflected) positions whereby they engage, e.g., snap into, the groove. In the illustrated embodiment, there is about 0.5 mm (per side) engagement of the fingers 113 with the groove 206 of the stem.

The stem 202 may preferably be larger than the lumen of the seal as shown in FIG. 8. As a result, when the fingers 113 are locked with the groove 206, the stem is sufficiently positioned within the seal 21 to ensure the formation of a tight fluidic compression and leak-resistant seal between the seal and the stem 202.

The boot 106 may then be slid toward the proximal end of the catheter 20 until it covers the retaining member 104 of the connector as shown in FIG. 8. Alternatively, the boot 106 could have been positioned over, e.g., assembled with, the housing 102 prior to coupling the catheter 20 to the stem, in which case the boot 106 would be positioned with the housing 102 during interconnection with the stem.

To remove the catheter 20 from the stem 202, the connector 100 may be actuated to disengage the locking fingers 113 from the stem as already described above. The boot 106 may include one or more features that assist with actuation of the connector. For example, as shown in FIGS. 3 and 4, the boot may include diametrically opposing flat surfaces 126 (only one shown) on its exterior. These flat surfaces may align with the horizontal tabs 117 of the retaining member, e.g., align with the major axis, thereby providing the physician with a tactile indication of where compressive force should be applied. In place of the flat surfaces 126, or in addition thereto, the boot 106 may also include visual markings 128, as shown in FIGS. 3 and 4, to indicate the correct location for the application of the compressive force. The compressive force necessary to actuate the connector may vary. However, in one embodiment, it is preferably about 1 to about 10 pounds-force (lbf) (about 4 to about 44 Newtons (N)), and, more preferably, about 5 to about 10 lbf (about 22 to about 44 N).

While the connector assemblies illustrated herein may be scaled for use with most any size of catheter, an exemplary infusion pump/catheter connector as described herein may utilize a housing 102 having an outer body diameter of about 5 to about 7 mm (e.g., about 5.8 mm) and a length of about 6 to about 8 mm (e.g., about 7.3 mm) for a catheter having a diameter of about 2 mm. The boot 106 may have an outer diameter of about 8 to about 10 mm (e.g., about 9.1 mm) and a length of about 10 to about 15 mm (e.g., about 12.5 mm). The overall volume of the connector 100 may be less than about 1 cubic centimeter (cc), preferably less than about 0.95 cc, and more preferably, less than about 0.65 cc.

Embodiments of the present invention provide a relatively low-profile catheter/pump connector that may be intuitively locked and unlocked without resort to the use of tools. Further, the connectors may assist in centering or redirecting a stem inserted off-axis such that a leak-resistant connection can be achieved between the stem and the catheter. Moreover, the components of the connector are captivated, reducing the risk of lost or misplaced parts. Still further, the snap-fit nature of the connector provides the physician with tactile and/or auditory feedback when the locking fingers engage the stem groove. Still further, connectors in accordance with embodiments of the present invention provide excellent resistance to catheter separation when subjected to anatomically-induced separating forces, yet may be unlocked (via clinician actuation) to permit removal with minimal pulling force.

Connectors, systems and methods in accordance with illustrative embodiments of the invention are provided herein and reference has been made to possible variations within the scope of the invention. These and other variations, combinations, and modifications will be apparent to those skilled in the art without departing from the scope of the invention, and it should be understood that this invention is not limited to the illustrative embodiments set forth herein. Accordingly, the invention is to be limited only by the claims provided below, and equivalents thereof.

What is claimed is:

1. A medical system comprising:
a fluid delivery device comprising a protruding stem; and
a catheter assembly comprising:
a catheter body defining a lumen;
a seal attached at or near a proximal end of the catheter body, the seal defining a seal opening in fluid communication with the lumen;
a connector surrounding the seal, the connector configured to secure an end of the catheter assembly to the stem; and
a guide ring surrounded by the connector and located between the seal and an end face opening of the connector, wherein the guide ring comprises a tapered inner surface configured to contact the stem and align a centerline of the seal opening with a centerline of the stem when the stem is received into the end face opening of the connector.

2. The system of claim 1, wherein the seal comprises a compression surface configured to contact an abutting surface of the connector, and wherein the compression surface lies within a plane perpendicular to the centerline of the seal opening.

3. The system of claim 1, wherein the guide ring is positioned proximate the seal.

4. The system of claim 1, wherein the guide ring comprises a material having a hardness greater than a hardness of the seal.

5. The system of claim 1, wherein the tapered inner surface defines a centerline coaxial with the centerline of the seal opening, the tapered inner surface tapering from a large diameter to a small diameter, the small diameter located proximate the seal.

6. The system of claim 1, wherein the connector comprises a retaining member having one or more locking members, the one or more locking members configured to engage a receiving portion of the stem to secure the catheter assembly relative to the stem.

7. The system of claim 6, wherein the connector further comprises a housing surrounded by the retaining member, the housing comprising a tubular wall defining a window operable to receive the one or more locking members.

8. A medical system comprising:
an implantable medical device comprising a protruding stem; and
a catheter assembly comprising:
a catheter body comprising a first end defining a ring-shaped seal, the seal defining a seal opening configured to receive therein the stem;
a tubular connector configured to surround at least the seal, the connector comprising a first end defining an end face opening, the seal being spaced-apart from the end face opening of the connector; and
an internal guide surface positioned within the connector between the end face opening and the seal, the guide surface comprising a material resistant to deformation or damage upon contact with the stem of the medical device, wherein the guide surface is configured to align a centerline of the stem, upon insertion of the stem into the end face opening of the connector, with a centerline of the seal opening.

9. The system of claim 8, wherein the connector further comprises:
a retaining member, the retaining member including:
a tubular sleeve comprising an elliptical cross-section defined by a major axis and an orthogonal minor axis; and
a locking finger positioned along the minor axis and extending inwardly from the sleeve, the retaining member deflectable from a first position to a second position, wherein the locking finger, when the retaining member is in the second position, is displaced outwardly from its location when the retaining member is in the first position; and
a housing surrounded by the retaining member, the housing comprising a tubular wall defining a window operable to receive the locking finger.

10. The system of claim 8, wherein the guide surface comprises a ring having an outer surface configured to seat with interference against an inner surface of a housing of the connector.

11. The system of claim 10, wherein the outer surface of the guide ring is tapered as compared to the inner surface of the housing.

12. The system of claim 8, wherein the guide surface comprises a guide surface material different than a material of one or both of the catheter body and the seal.

13. The system of claim 8, wherein the guide surface is frusto-conical and defines a cone angle of about 70 degrees to about 90 degrees.

14. The system of claim 8, wherein the seal comprises a flange defining a compression surface and the connector defines an abutting surface configured to contact the compression surface when the connector is secured to the stem.

15. The system of claim 14, wherein the compression surface lies within a plane perpendicular to the centerline of the seal opening.

16. A method for connecting a catheter to a medical device, the method comprising:
receiving a stem of the medical device into an opening of a connector attached to a first end of the catheter, the connector comprising:
a housing comprising a first end defining the opening, the housing surrounding a ring-shaped seal spaced-apart from the first end, the seal attached to the catheter and defining a seal opening configured to receive therein the stem of the medical device; and
a tapered guide surface positioned within the housing between the first end and the seal;
introducing the stem of the medical device into the opening of the housing such that a longitudinal axis of the stem is misaligned from a longitudinal axis of the connector;
contacting the stem of the medical device with the guide surface; and
sliding the stem along the guide surface until the longitudinal axis of the connector is aligned with the longitudinal axis of the stem.

17. The method of claim 16, further comprising engaging one or more locking members associated with the connector with a receiving portion of the stem.

18. The method of claim 16, further comprising surrounding the connector with a boot.

19. The method of claim 16, further comprising locating the guide surface to minimize or prevent contact between the stem and the seal.
